(19) United States
(12) Patent Application Publication
(10) Pub. No.: US 2023/0323300 A1
(43) Pub. Date: Oct. 12, 2023

(54) METHOD FOR IN VITRO PRODUCTION OF RED BLOOD CELLS

(71) Applicant: IUCF-HYU (INDUSTRY-UNIVERSITY COOPERATION FOUNDATION HANYANG UNIVERSITY), Seoul (KR)

(72) Inventors: Eun Jung BAEK, Seoul (KR); Eun MI LEE, Hanam-si (KR); So Yeon HAN, Asan-si (KR); Won-Seok CHOI, Incheon (KR); Jang Han LEE, Daejeon (KR); Hyo Sang LEE, Sejong-si (KR)

(73) Assignee: IUCF-HYU (INDUSTRY-UNIVERSITY COOPERATION FOUNDATION HANYANG UNIVERSITY), Seoul (KR)

(21) Appl. No.: 18/022,415

(22) PCT Filed: Aug. 17, 2021
(86) PCT No.: PCT/KR2021/010880

§ 371 Date: Feb. 21, 2023

(30) Foreign Application Priority Data
Aug. 21, 2020 (KR) 10-2020-0105419

(51) Int. Cl. C12N 5/078; C12M 3/06

(52) U.S. Cl. CPC C12N 5/0641; C12M 27/14; C12N 2527/00; C12N 2506/11

(57) ABSTRACT

The present invention relates to a method for in vitro production of red blood cells. The method for in vitro production of red blood cells, according to the present invention, specifies, by cell size, the maturation step of cells exhibiting optimal effects in an agitation-type culture, so that even if an expert does not always identify cell shape, automated processes of culturing are possible, and thus automation in bioreactors is possible in the mass-production of uniform quality and red blood cells.

### TECHNICAL FIELD

The present invention relates to a method for in vitro production of red blood cells.

### BACKGROUND ART

Due to the shortage of blood for transfusion, there is an increasing demand for a technique for producing red blood cells from stem cells in vitro. In Korea, about 2.1 million units of red blood cell preparations are used annually, but when it is calculated that there are 2×10¹² red blood cells per unit, about 4×10¹⁸ or more red blood cells per year are needed. In the case of the United States, there is a need for 10¹⁹ or more red blood cells. Although 80 million units of collected blood are supplied worldwide every year, it is greatly insufficient to meet the demand.

Further, since red blood cell reagents used for irregular antibody screening & identification tests in transfusion medicine are also manufactured by receiving blood supplied from blood donors, it is difficult to produce and supply the reagents.

In order to commercialize a technique for in vitro culture of red blood cells to overcome various side effects and contamination and infection of blood provided by blood donors or difficulties in supply, it should be possible to automate a process capable of exhibiting an optimal red blood cell yield suitable for mass production of red blood cells of uniform quality under constant conditions. Therefore, it is essential to establish optimal culture conditions (parameters) and develop a culture technique using a bioreactor such that consistent results can be obtained.

### DISCLOSURE

### Technical Problem

An object of the present invention is to provide a method for in vitro production of red blood cells.

### Technical Solution

Accordingly, the present inventors have conducted various studies to establish optimal culture conditions that enable automated mass production of red blood cells using a bioreactor. As a result, it could be confirmed that during culturing of erythroid progenitor cells, by specifying the time point of conversion from stationary culture to agitation culture based on the diameter of the erythroid cells during culture, red blood cells can be efficiently produced in vitro from erythrocyte progenitor cells regardless of a culture environment such as agitation speed and without co-culturing with supporting stromal cells. That is, the present invention provides a method for preparing red blood cells at high yield by performing stationary culture in a culture vessel in an environment with a medium composition that does not include stromal cells, and converting the erythrocyte progenitor cells to agitation culture at a specific time point.

Accordingly, the present invention provides a method for in vitro production of red blood cells, including the step of converting the erythrocyte progenitor cells into agitation culture during culturing of the erythrocyte progenitor cells.

Hereinafter, the configuration of the present invention will be described in detail.

The present invention provides a method for in vitro production of red blood cells, including the step of, during the culture of erythrocyte progenitor cells, converting the cultured cells into agitation culture when the diameter of the erythrocyte progenitor cells reaches 10 to 15 μm.

First, the method for in vitro production of red blood cells of the present invention may be performed in a culture vessel. The culture vessel refers to a culture vessel capable of controlling a culture environment such as agitation speed, temperature, dissolved oxygen (DO), or pH according to the culture mode of cells, that is, stationary culture and agitation culture.

In the present invention, “progenitor cells” refers to undifferentiated cells having self-renewal ability and differentiation potency, but ultimately differentiated cells whose types of finally differentiated cells have already been determined. Although progenitor cells are committed to a differentiation pathway, they generally do not express markers of mature, fully differentiated cells or function as mature, fully differentiated cells. Therefore, although progenitor cells differentiate into related cell types, they cannot form a wide variety of cell types in a normal state. In the present invention, erythrocyte progenitor cells are used.

As used herein, “differentiation” refers to a phenomenon in which a structure or function is specialized while cells divide, proliferate, and grow, that is, means that the morphology or function of cells, tissues, and the like of an organism changes in order to carry out the tasks given to them. In general, differentiation is a phenomenon in which a relatively simple system is separated into two or more qualitatively distinct subsystems. For example, as in the distinction of the head and body between the egg parts that were initially homogeneous in ontogeny, or the distinction of cells such as muscle cells and nerve cells even in cells, a state of the development of qualitative differences between initially nearly homogeneous parts of a biological system, or the resulting separation into qualitatively distinct regions or subsystems is called differentiation.

In the present invention, erythrocyte progenitor cells may be obtained from various sources such as peripheral blood, cord blood, or bone marrow. The erythrocyte progenitor cells may be erythroid cells before enucleation. In addition, the erythrocyte progenitor cells may be proerythroblasts, basophilic erythroblasts, polychromatic erythroblasts, orthochromatic erythrocytes, or mixtures thereof.

In one embodiment, the erythrocyte progenitor cells may be CD71 positive (CD71+) cells or glycophorin A positive (GPA+) cells, and may be cells differentiated from hematopoietic stem cells into erythroid cells by treatment with erythropoietin. CD71+ cells or GPA+ cells as erythrocyte progenitor cells may be isolated by various cell isolation methods known in the art, for example, an immunomagnetic-bead isolation method using CD71+ antibodies.

In one embodiment, the erythrocyte progenitor cells may be cells derived from cord blood, bone marrow or peripheral blood.

Erythrocyte progenitor cells differentiate into mature erythrocytes through the erythropoiesis process consisting of the following stages: (a) differentiation from hematopoietic stem cells into proerythroblasts; (b) differentiation from proerythroblasts into basophilic erythroblasts; (c) differentiation from basophilic erythroblasts into polychromatic erythroblasts; (e) differentiation from polychromatic erythroblasts into orthochromatic erythroblasts; and (f) differentiation from orthochromatic erythroblasts to erythrocytes (red blood cells) via reticulocytes.

In the method according to the present invention, the erythrocyte progenitor cells may be cultured by an appropriate method known in the art or a modified method thereof. More specifically, the erythrocyte progenitor cells may be cultured in a stroma-free medium. As the stroma-free medium, any medium capable of growing blood cells may be used as a basic medium, and for example, Iscove's modified dextrose media (IMDM); Stemline II hematopoietic stem cell expansion media (Sigma) specialized for blood cells; X-vivo media (Lonza, M D, USA), or Stemspan media (Stem Cell Technologies) may be used. Reagents added to the media are not very limited, and for example, reagents used in [Baek E J, Kim H S et al. 2009] may be included, but are not limited thereto. In one embodiment of the present invention, the medium may be a serum-free, plasma-free and stroma-free medium including albumin, transferrin, ferric nitrate, insulin, L-glutamine and monothioglycerol, that is, a Stemline II hematopoietic stem cell expansion medium or Iscove's modified dextrose media containing the aforementioned components.

Furthermore, the medium used in the present invention may include vitamin C so as to maintain a stable state against oxidative stress in vitro, may further include an antibiotic such as penicillin, streptomycin or gentamycin, if necessary, and may further include at least one among a stem cell factor, interleukin-1, interleukin-3, interleukin-4, interleukin-5, interleukin-11, a granulocyte macrophage colony stimulating factor, a macrophage colony stimulating factor, a granulocyte colony stimulating factor or erythropoietin.

The method of the present invention includes the step of, during the culture of erythrocyte progenitor cells, converting the cultured cells into an agitation culture type when the diameter of the erythrocyte progenitor cells reaches 10 to 15 μm, for example, 11 to 14 μm, and 12 to 13 μm.

In the above step, the erythroid cells when converted into the agitation culture type may include erythroid cells in the growth and maturation phases.

As in the present invention, when the diameter of erythrocyte progenitor cells reaches a range of 10 to 15 μm, in the case where the culture cells are converted into the agitation culture type, cells with the diameter size as described above have reached the maturation phase, so that in this case, when the agitation culture type is applied, not only cell proliferation occurs well, but also excellent cell viability and an excellent enucleation rate may be exhibited.

In the present invention, erythrocyte progenitor cells may be stationary-cultured in a culture vessel before the diameter of erythrocyte progenitor cells reaches a range of 10 to 15 μm.

In the present invention, the stationary culture refers to culturing in a state of being allowed to remain in a culture vessel without agitating or shaking, is performed under conditions free from hydrodynamic shear stress except when media are exchanged, and is also called 2D culture or planar culture. However, stationary culture in the present invention may also include those performed in bioreactors or agitators. In this case, the “free from hydrodynamic shear stress” means that the hydrodynamic shear stress is substantially an agitation speed of less than 30 rpm or a tip speed of less than 0.018 m/s. For example, it may mean that the hydrodynamic shear stress during the stationary culture is substantially an agitation speed of less than 10 rpm or a tip speed of less than 0.006 m/s.

In the present invention, the tip speed may be determined by the revolutions per minute (rpm) of a bioreactor or agitator and the diameter of a rotating blade, and can be specifically shown in Table 1 according to the following General Formula 1.

Tip speed=Revolutions per minute (rpm)×0.262×Diameter of rotating blade (inch) [General Formula]

TABLE 1

| Revolutions | | | |
|---|---|---|---|
| per minute | | Rotating blade | |
| (rpm) | 0.262 | diameter (inch) | Tip speed |
| 10 | 0.262 | 0.00229 | 0.006 |
| 30 | 0.262 | 0.00229 | 0.018 |
| 50 | 0.262 | 0.00229 | 0.03 |
| 300 | 0.262 | 0.00229 | 0.18 |
| 400 | 0.262 | 0.00229 | 0.24 |
| 450 | 0.262 | 0.00229 | 0.27 |
| 500 | 0.262 | 0.00229 | 0.3 |
| 600 | 0.262 | 0.00229 | 0.36 |
| 700 | 0.262 | 0.00229 | 0.42 |
| 800 | 0.262 | 0.00229 | 0.48 |

Since it has been described in many documents that the tip speed is proportional to the hydrodynamic shear stress (Kim Gail Clarke., Bioprocess Engineering, 9—Bioprocess scale up, 2013, pages 171-188), the description of the hydrodynamic shear stress in the present invention in terms of tip speed and agitation speed (or revolutions per minute) will correspond to the self-obvious level for those skilled in the art. In the present invention, the stationary culture may be performed at a temperature of 20 to 38° C., for example, 30 to 37° C., but is not limited thereto.

A culture vessel that can be used for the stationary culture used in the present invention may include a flask, a T-flask, a disposable cell culture bag, an agitator or a bioreactor, but is not limited thereto.

In the present invention, agitation culture refers to 3D culture (three-dimensional culture), and may be performed under conditions where there is hydrodynamic shear stress due to medium flow. In this case, the “there is a hydrodynamic shear stress” means that the hydrodynamic shear stress is an agitation speed of 10 rpm or more or a tip speed of 0.006 m/s or more. Further, the agitation culture (3D culture) is a method capable of culturing cells at a high cell concentration per standard volume such that cells can be accumulated in one or more layers, and agitation culture may involve culturing cells at high density in order to efficiently utilize the space and medium required for cell culture. The agitation culture may increase the cell density of erythroid cells by smoothly supplying oxygen and nutrients to the cells through agitation of the medium. For example, the cell density may be in a range of 1×10⁴ cells/mL to 5×10⁷ cells/mL, more preferably 1×10⁵ cells/mL to 1×10⁷ cells/mL, and for example, 0.5×10⁶ cells/mL to 5×10⁶ cells/mL. When erythroid cells are cultured at the cell density as described above, the proliferation rate and enucleation rate of erythroid cells may be very high. In addition, the agitation culture may be performed at a temperature condition of 28 to 38° C., for example, 30 to 37° C., depending on the cell maturation period, but is not limited thereto. Furthermore, in the agitation culture, cells may be cultured at an agitation speed of 50 to 700 rpm, for example, 100 to 650 rpm, 200 to 650 rpm, and 300 to 600 rpm, and may be cultured at a tip speed of 0.03 m/s to 0.42 m/s, for example, 0.06 m/s to 0.39 m/s, 0.12 m/s to 0.39 m/s, and 0.18 m/s to 0.36 m/s. In this case, in agitation culture, erythroid cells before enucleation may be agitation-cultured at 35 to 38° C., and when the enucleation process in in vitro culture is imminent, cell death (apoptosis) of erythroid cells increases and cell viability drops sharply, but in vitro culture may be performed at 28 to 33° C. after 15 days of culture because the maturation of erythroid cells may be stabilized by lowering the culture temperature (Kim H O & Baek E J., Tissue engineering part A, 2012; 18 (1-2):117-26.).

In one embodiment, red blood cells produced according to the present invention were found to have a GPA+/CD71−/nuclei-type similar to that of fresh peripheral blood red blood cells (PB RBC). This means that the red blood cells according to the present invention correspond to mature red blood cells. Further, it was confirmed that the red blood cells produced according to the present invention were successfully stored in a refrigerator for 21 days and most of the red blood cells maintained their biconcave red blood cell shape well (Example 3 and FIG. 9).

Therefore, the agitation culture according to the present invention may be for obtaining fully mature red blood cells.

A reaction vessel that can be used for the agitation culture used in the present invention may include a shaking flask, a shaking incubator, a fermentation tank, a T-flask, a disposable cell culture bag, a bioreactor, but is not limited thereto.

In one embodiment, the reactor may be a bioreactor.

In one embodiment, the agitation culture may be performed under conditions of a temperature of 30 to 37° C., a dissolved oxygen (DO) of 10 to 50%, and an agitation speed of 250 to 500 rpm or a tip speed of 0.15 to 0.30 m/s.

The reactor for stationary culture and the reactor for agitation culture may be the same or different. For example, when the reactor for stationary culture and the reactor for agitation culture are the same, after the stationary culture is completed in the same reactor, cells can be cultured by an agitation culture method by additionally supplying a medium including necessary nutrients such as cytokines. In addition, when the reactor for stationary culture and the reactor for agitation culture are different, after the stationary culture is completed, a culture may be transferred to the reactor for agitation culture to perform the agitation culture.

In conventional studies to produce red blood cells in vitro, red blood cells have been produced by culturing under sparging-free constant conditions under conditions of a pH of 7.5, a dissolved oxygen (DO) of 50% and 450 rpm (0.27 m/s) in a microbioreactor until hematopoietic stem cells become red blood cells. However, in the case of conventional studies, the step of manually confirming the shape and condition of the cells by an expert during the process of producing red blood cells was essentially performed, and even with this, there was no objective indication of when and under what conditions it should be placed in the incubator, so that it was difficult to perform automated process culture. However, the method according to the present invention divides the in vitro red blood cell production process into maturation phases (growth phase, maturation phase, and enucleation phase) with different cell properties to establish detailed process conditions for each phase, and optimal culture conditions for each subdivided phase were derived. The method for in vitro production of red blood cells according to the present invention enables automated process culture without the need for an expert to confirm the shape of cells each time by specifying the maturation phase of the cells, which exhibits the optimum effect in agitation culture by the cell size.

Furthermore, the present invention provides a blood preparation including red blood cells produced by the method for in vitro production of red blood cells as described above.

In the following examples, it was confirmed that the red blood cells produced in vitro by the method of the present invention not only have an excellent oxygen-carrying capacity similar to that of freshly donated blood, but also have an excellent degree of deformation under pressure compared to general red blood cells, and thus, red blood cells have the same level of function as red blood cells.

Therefore, red blood cells produced according to the present invention may have various therapeutic actions.

In one embodiment, the red blood cells may be used for transfusion. The ability to produce large amounts of cells for transfusion can alleviate the chronic shortage of blood experienced by blood banks and hospitals nationwide. The method of the present invention allows the production of universal cells for transfusion.

A specific aspect of the present invention relates to the expansion of human red blood cells to reach commercial amounts.

Human red blood cells are produced on a large scale, stored as needed, and supplied to hospitals, clinicians, or other health care facilities. Once a patient shows indications such as, for example, ischemia or vascular injury, or requires hematopoietic reconstitution, human red blood cells may be ordered and supplied in a timely manner. Therefore, the present invention relates to a method for generating and expanding human red blood cells to reach commercial scale, a cell preparation including human red blood cells derived from the above method, and a method for providing human red blood cells to hospitals and clinicians (that is, producing, optionally storing and selling).

Additionally, another specific aspect of the present invention relates to a method for producing, storing and distributing red blood cells produced by the method described in the present invention. After the generation and expansion of human red blood cells ex vivo or in vitro, the human red blood cells may be harvested, purified, and optionally stored prior to patient treatment. Therefore, in one embodiment, the present invention provides a method for supplying red blood cells to hospitals, health care centers, and clinicians, whereby red blood cells produced by the method described in the present invention are stored, ordered on demand by hospitals, health care centers or clinicians, and administered to patients in need of red blood cell therapy.

The benefits and features of the present invention, and the methods of achieving the benefits and features will become apparent with reference to embodiments to be described below in detail. However, the present invention is not limited to the exemplary embodiments to be disclosed below, and may be implemented in various other forms, and the present exemplary embodiments are only provided for rendering the disclosure of the present invention complete and for fully representing the scope of the invention to a person with ordinary skill in the technical field to which the present invention pertains, and the present invention will be defined only by the scope of the claims.

### Advantageous Effects

The present invention relates to a method for in vitro production of red blood cells. The method for in vitro production of red blood cells, according to the present invention, specifies, by cell size, the maturation stage of cells exhibiting optimal effects in an agitation-type culture to select the time point of an agitation culture, so that even if an expert does not always identify the cell shape, automated processes of culturing are possible, and thus automation in bioreactors is possible in the mass-production of uniform quality and red blood cells.

### DESCRIPTION OF DRAWINGS

FIG. 1 illustrates a schematic view of the phasewise growth process of erythroid cells and the process steps optimized for the production of enucleated red blood cells.

FIG. 2 illustrates the characteristics of erythroid cells cultured in agitation culture by inoculating erythroid progenitor cells at an immature phase into a bioreactor on day 7 of stationary culture, A shows the results of confirming viable cell density (VCD), cell viability and cell diameter by rpm, and B shows the observation of the cell shape by rpm for each day of culture.

FIG. 3 illustrates the characteristics of erythroid cells cultured in agitation culture by inoculating erythroid progenitor cells at an immature phase into a bioreactor on day 7 of stationary culture, A shows the results of confirming viable cell density, cell viability and cell diameter according to the medium composition; B shows the percentage of erythroid cells among cultured cells according to the medium composition; and C shows the results of observing cells cultured according to the medium composition by Wright-Giemsa staining.

FIG. 4 confirms cell characteristics in agitation culture by inoculating erythroid cells at the growth and maturation stages into a bioreactor on day 12 (D12+0) of stationary culture, A shows viable cell density (VCD); B shows the enucleation rate; C shows the observation of cells in which agitation culture is initiated on day 12 of stationary culture of erythroid cells at the growth and maturation stages, enucleated red blood cells are indicated with arrows; and D shows the percentage of erythroid cells of cells cultured according to the medium composition.

FIG. 5 confirms the cell characteristics in agitation culture by inoculating erythroid cells at maturation phase into a bioreactor on day 10, day 11, day 12 and day 13 based on the stationary culture day, respectively, A shows the change in cell diameter during the culture period by cell inoculation day in the bioreactor; B shows the change in cell diameter by cell inoculation day; C shows the cell viability during the culture period by cell inoculation day; D shows the enucleation rate after final culture by cell inoculation day; E shows the cell expansion fold during the culture period by cell inoculation day; F shows the amount of red blood cells obtained after final culture by cell inoculation day; G shows the degree of cell maturation change during the culture period by inoculation day in terms of the ratio of erythroid cell types; and H is a set of photographs of Wright-Giemsa-stained cells obtained on the final culture day (day 18 of culture) by cell inoculation day, and arrows indicate enucleated red blood cells.

FIG. 6 illustrates the results of confirming the optimal conditions using DoE when erythroid cells at the growth stage are cultured in a bioreactor, A shows the conditions of the culture process; B shows the fit and significance analysis of the DoE model for the highest viable cell concentration (pVCD) during the culture period and the significant values of each parameter; C shows the fit and significance analysis of each DoE model for the conditions showing the highest number of cells with polychromatic and orthochromatic erythroblasts and significant values for each parameter; and D shows a graph evaluating the correlation between process results and process factors of the DoE model.

FIG. 7A shows the viable cell density according to the inoculation conditions of a bioreactor; B shows the viable cell density according to the inoculation conditions of a bioreactor in consideration of the actual cell proliferation rate; C shows the red blood cell yield according to the inoculation day of the bioreactor; D shows the red blood cell yield according to the inoculation concentration; E shows the cell viability according to the inoculation concentration; and F shows the cell diameter according to the inoculation day.

FIG. 8 illustrates the functional evaluation results of red blood cells produced by the method according to the present invention, A shows the observation of the shape after performing pure red blood cell filtration with a filter; B shows the results of confirming red blood cell deformability by applying pressure to red blood cells; and C shows the results of confirming the oxygen-carrying capacity of red blood cells.

FIG. 9 illustrates the functional evaluation results of red blood cells produced by the method according to the present invention, A shows the results of observing the shape of red blood cells according to the refrigerated storage; B shows the results of the blood type test; C confirms the degree of hemoglobin expression; and D shows the results of analyzing red blood cells (bioreactor-RBC) with attached fluorescent antibodies against GPA, CD71 and nuclei using flow cytometry to confirm that they are mature red blood cells.

Hereinafter, the present application will be described in detail through examples. The following examples only illustrate the present application, and the scope of the present application is not limited to the following examples

### MODES OF THE INVENTION

### Examples

### Example 1 Agitation Culture of Erythroid Cells for Each Phase of Erythropoiesis

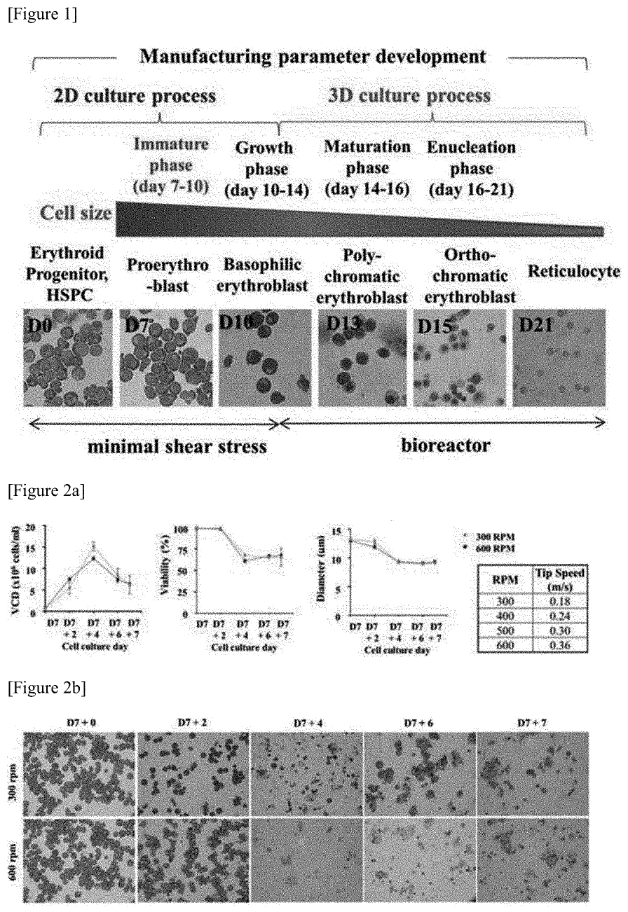

Since erythroid cells at the immature phase are vulnerable to shearing stress, the artificial erythrocyte production culture using hematopoietic stem cells or hematopoietic precursor cells is performed not by an agitation method but by a weak medium flow method. Examples of the weak medium flow method include a method of initiating stationary culture (2D culture process) on a cell culture plate, and then changing to an agitation culture process when the cell number and cell concentration increase. In this case, the corresponding process may be divided into an immature phase, a growth phase, a maturation phase and an enucleation phase, and may be shown as a detailed process for each phase (FIG. 1). In this case, the culture days in parentheses in FIG. 1 are the culture days corresponding to the production of erythroid cells in cord blood hematopoietic stem cells, and the erythroid cells become smaller in size as they mature. In order to automate mass production in an apparatus (device), an appropriate culture method for each detailed step and an automatic measurement and reading method for moving to the next step are required, and in the present example, it was intended to clarify whether it is possible to confirm the size of erythroid cells in culture through monitoring.

The development of the culture process was performed by establishing a process plan using the design of experiment technique, and the effectiveness of a model was confirmed through statistical analysis of the correlation between process results and process factors, and an optimum value was derived. After switching to a bioreactor, the optimization of process conditions was performed for each step during the continuous culture process.

FIG. 1 illustrates a schematic view of the process step optimized for the phasewise growth process of erythroblasts and the process steps optimized for the production of enucleated red blood cells. In this case, the cytokines and reagents put in the media for each period followed what is described in the [Kim S H et al, (2019) “Improvement of Red Blood Cell Maturation In Vitro by Serum-Free Medium Optimization.” Tissue Eng Part C Methods 25 (4): 232-242] document.

1) Agitation Culture of Erythroid Progenitor Cells at Immature Phase

Frozen cord blood hematopoietic stem cells (CD34+ cells) were cultured in a Stemline II medium (Sigma Aldrich, St Louis, MO), which is a hematopoietic stem cell proliferation medium, in a 2D flask in a 5% CO₂ incubator at 37° C. and a cell inoculation concentration of 1×10⁵ cells/mL, and after the hematopoietic stem cells were cultured, proerythroblasts were put into a continuously stirred-tank bioreactor (Ambr™, Sartorius) on day 7 of culture day, and cultured under agitation at 300 rpm and 600 rpm for 7 days, respectively. In this case, the medium was replaced every two days (50% ratio) while performing agitation culture under conditions of 37° C., a cell inoculation concentration of 0.5×10⁶ cells/mL, a cell solubility of 25%, and a pH of 7.4. For cultured cells, viable cell density (VCD), cell viability and cell diameter were measured (FIG. 2A). In this case, cell viability was measured by counting only viable cells using trypan blue staining. Further, comparisons were made through the annexin V assay by a flow cytometry method. That is, cells were stained with annexin V-PE and propidium iodide (PI: Invitrogen, Camarillo, CA) at room temperature for 15 minutes according to the manufacturer's instructions. The cells were washed twice with a wash buffer and then analyzed using flow cytometry.

As a result, there was no significant difference in viable cell density (VCD), cell viability and diameter according to rpm (FIG. 2A; mean+standard deviation). On day 7 of culture, agitation culture was initiated by inoculating cells into a bioreactor, and then after 4 days (D7+4), erythroid progenitor cells were centrifuged at 300 rpm and 600 rpm, and as a result, it could be seen that erythroid progenitor cells are not suitable for agitation culture considering that almost all cells were dead at both speeds and only other lineage cells such as macrophages remained (FIG. 2B).

Next, in order to investigate whether the culture environment can be improved according to the medium, erythroid progenitor cells were cultured under various cell concentration and medium conditions ([Stemline II+5% plasma]; [Stemline II 80%+DEF-CS (Cellartis DEF-CS 500 Basal Medium, Takara, Kyoto, Japan) 20% (optimized mixed medium (OMM))]; and [Stemline II+DEF-CS™ XENO-FREE GMP Grade Basal Medium (Takara) (OMM (GMP (Good Manufacturing Practice))]. The specific culture conditions are as follows: cells were inoculated at concentrations of 0.5×10⁵ cells/mL and 1.0×10⁶ cells/mL for each medium, and cultured under conditions of 37° C., a pH of 7.4, 300 rpm, and a dissolved oxygen of 25%, and the medium was replaced every 2 days (50% ratio). In this case, OMM means that Stemline : DEF-CS were mixed at a volume ratio of 8:2, and the reagents added to the medium are as follows:
150 mg/mL holo-transferrin; 90 ng/mL ferric nitrate; 30.8 mM vitamin C; 160 mM 1-thioglycerol; 50 mg/mL insulin; 4 mM 1-glutamine; 2 mg/mL cholesterol; 0.05% Pluronic F-68; and 0.5 mL/mL lipid mixture.

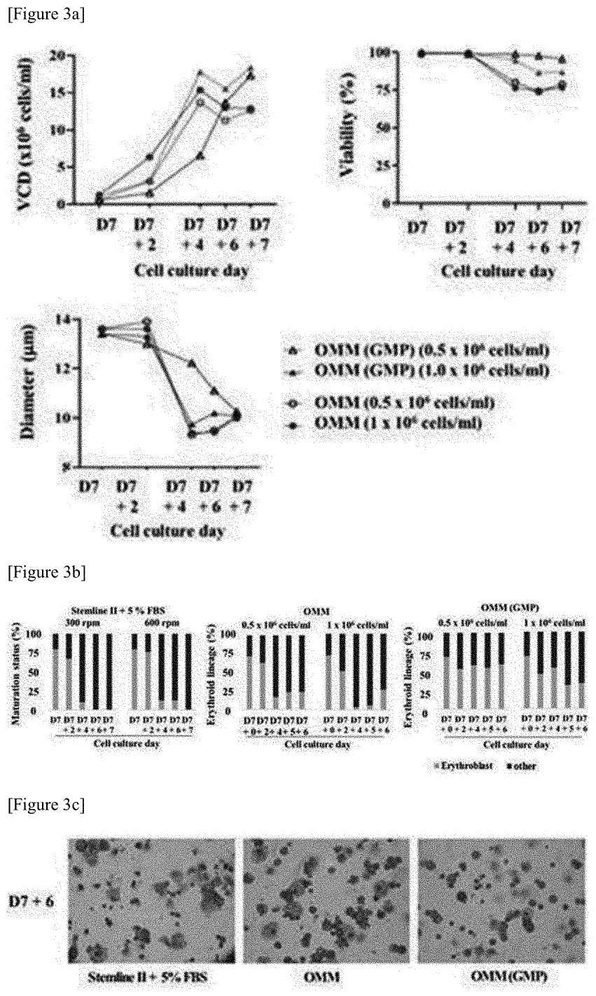

As a result, it could be confirmed that similar to previous experimental results, erythroid progenitor cells at the immature phase could not survive even under various culture conditions (FIGS. 3A and 3B), and after agitation culture was initiated by inoculation of Wright Giemsa stained-cells into the bioreactor, a large amount of non-erythroid progenitor cells were observed among the cells after 4 days (D7+4) (FIG. 3C).

2) Agitation Culture of Erythroid Cells at Growth and Maturation Phases

In stationary culture (2D culture), erythroid cells cultured under the conditions of [Stemline II+5% FBS] were inoculated into a bioreactor on day 12 of culture (D12) in which basophilic erythroblasts and polychromatic erythroblasts were mixed, and cultured under the [Stemline II+DEF-CS™ XENO-FREE GMP Grade Basal Medium (OMM (GMP))] medium conditions (37° C., a dissolved oxygen of 25%, pH 7.4, a cell inoculation concentration of 1.5×10⁶ cells/mL, 300 rpm, and 50% medium exchange every two days).

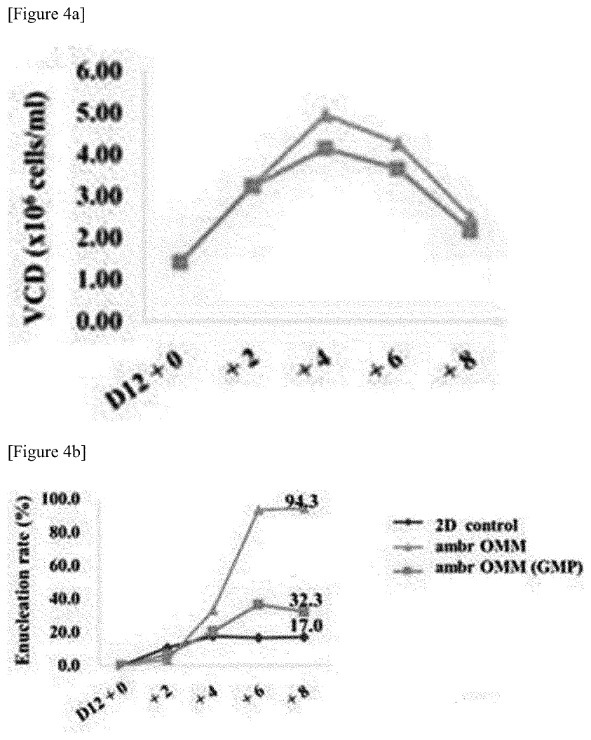

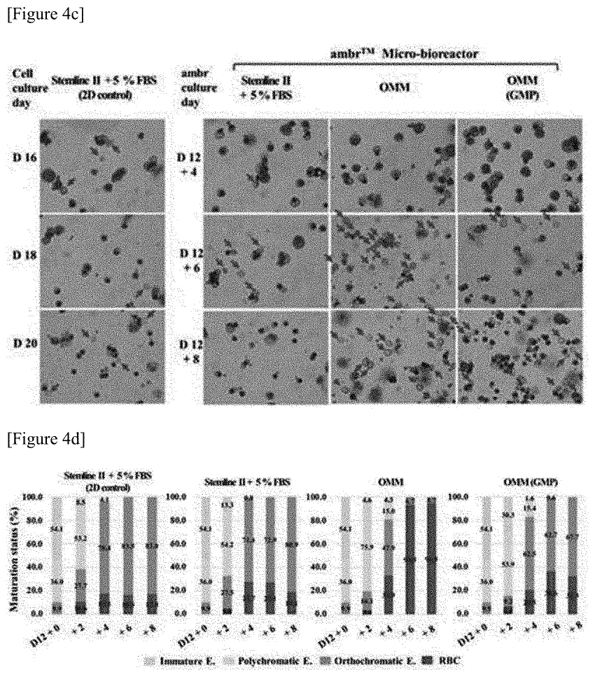

As a result, in all the resulting data, erythroid cells matured and grew well, and although there was no significant difference in cell viability or diameter between media, the density (VCD) was higher and the enucleation rate was very high at 94.3% particularly during culture in the OMM medium (Ambr OMM) (FIGS. 4A and 4B). This can also be seen in the cell observation results in FIG. 4C, where many enucleated red blood cells (arrows) were observed in the optimized mixed medium (OMM). In particular, after the agitator culture was initiated by inoculating the cells into the bioreactor under conditions of an optimized mixed medium (OMM), the maturation and enucleation into red blood cells continued to increase from day 4 (+4), and a high red blood cell yield was maintained until the final day of culture (FIG. 4D).

3) Agitation Culture of Erythroid Cells at Proliferation Phase

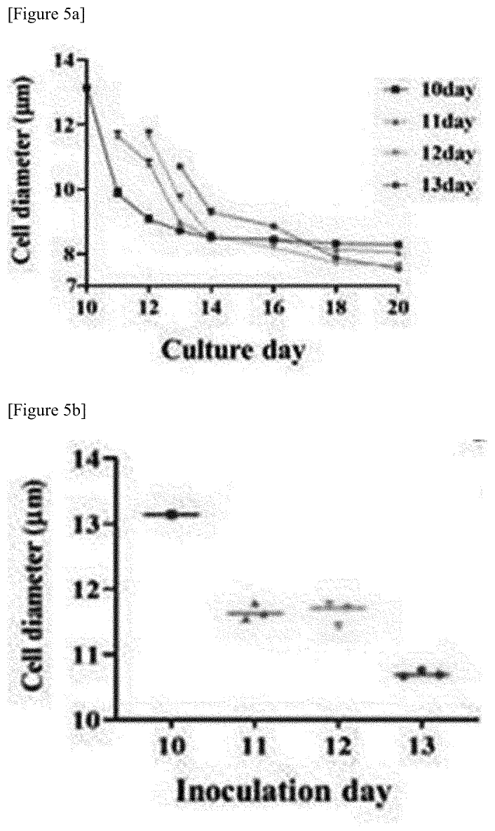

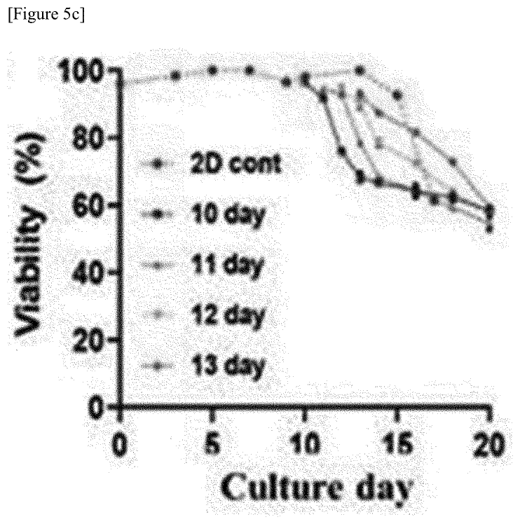

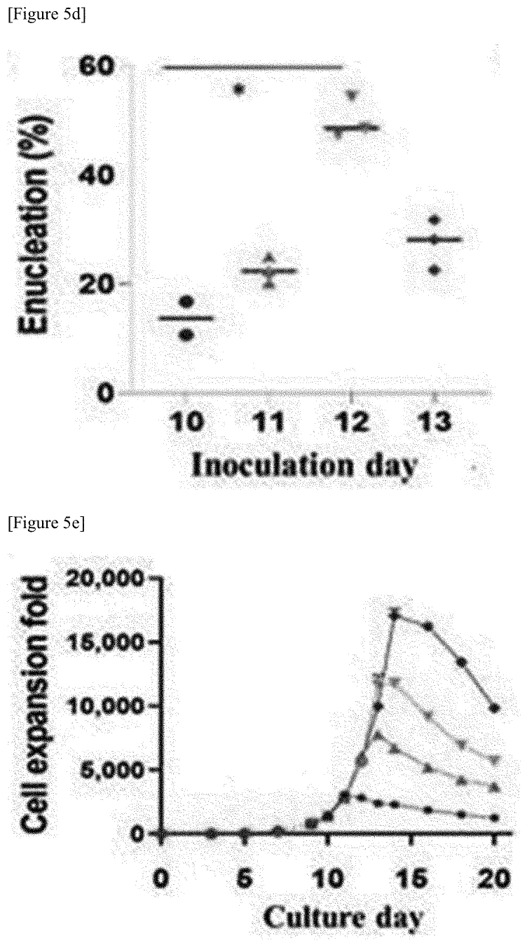

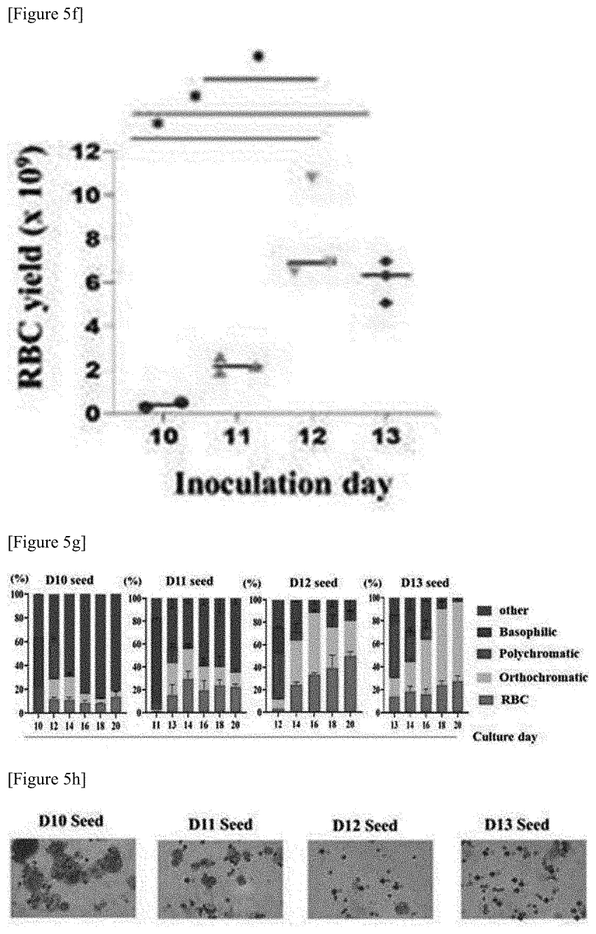

In the previous experiment 1), erythroid progenitor cells at the immature phase were cultured in stationary culture for 6 days, and then agitation culture was initiated on day 7 (D7) of culture, and in the experiment 2), erythroid cells at the growth and maturation phases were cultured in stationary culture for 11 days, and then agitation culture was initiated on day 12 of culture (D12). Accordingly, in order to specify the starting point of agitation culture of erythroid cells, erythroid progenitor cells were cultured in stationary culture for 9 to 12 days, respectively, and then agitation culture was initiated by inoculation into a bioreactor on day 10 (D10), day 11 (D11), day 12 (D12) and day 13 (D13) of stationary culture. As a result, in the case of erythroid cells, which were inoculated into the bioreactor on day 10 of culture to initiate agitation culture, the cell diameter, which showed an average of 13.14 μm, decreased very rapidly in one day, this was interpreted as erythroid cells at the immature phase quickly died in an agitated environment (FIG. 5A).

In addition, through the tendency of cell size at each inoculation time to decrease, it was understood that cells inoculated in the bioreactor on days 11 and 12 showed similar cell size results because cell proliferation occurred at this time (FIG. 5B). At this time, it seems that the later the agitation culture is initiated, the higher the cell viability can be maintained (FIG. 5C), and the enucleation rate was shown to be the highest when inoculated into the bioreactor on day 12 of culture (FIG. 5D). When the cumulative cell expansion fold was calculated from the very first single hematopoietic stem cell, it was found that the cell expanded best when it was inoculated late into the bioreactor (that is, when the agitation culture was initiated at the latest time) (FIG. 5E), and it could be seen that the number of red blood cells actually finally obtained by multiplying this value by the enucleation rate was the most significantly higher when inoculated into the bioreactor on day 12 of culture (FIG. 5F, ANOVA analysis, Kruskal_Wallis test, *P value<0.05). This can also be confirmed from the ratio of erythroid cells by type during the culture period for each day of inoculation of cells into the bioreactor, and the highest percentage of red blood cells was observed when inoculated into the bioreactor on day 12 of culture (FIG. 5G).

Furthermore, FIG. 5H is a set of representative slide photographs of Wright Giemsa-stained cells on the final culture day (day 18) of cells cultured for each cell inoculation day, and red arrows indicate enucleated red blood cells. From FIG. 5H, it could be seen that many enucleated red blood cells were generated from the cells inoculated into the bioreactor on days 12 and 13 of culture.

### Example 2 Confirmation of Optimal Conditions Using DoE when Erythroid Cells at Proliferation Phase are Cultured in Bioreactor

The conditions for in vitro production of red blood cells presented in the present invention were performed by designing experimental conditions based on the statistics-based design of experiment (DoE), and the present invention derived optimal process conditions by deriving correlations between experimental results and process factors as multivariate statistical functions.

1) Determination of Cell Inoculation Day and Inoculated Cell Concentration During Agitation Culture (Growth of Basophilic Erythroblasts and Polychromatic Erythroblasts)

Erythroid cells at the maturation phase (basophilic erythroblasts, polychromatic erythroblasts, orthochromatic erythroblasts and erythrocytes) that had reached culture days 12, 13 and 14 in stationary culture were inoculated into a bioreactor, and cultured on an OMM medium. The erythroid cells were cultured under conditions of 37° C., a dissolved oxygen (DO) of 25%, and a pH of 7.4 with 50% medium exchange every two days, and process results were evaluated by enumerating viable cell density (VCD), cell viability, cell diameter and polychromatic red blood cells and orthochromatic erythroblasts. In order to evaluate process results during culture, viable cell density (VCD), cell viability and cell diameter were measured by reading approximately 4,000 to 5,500 cells per sample measurement using Vi-Cell XR (Beckman Coulter, Miami, Florida, USA) for sampled cells.

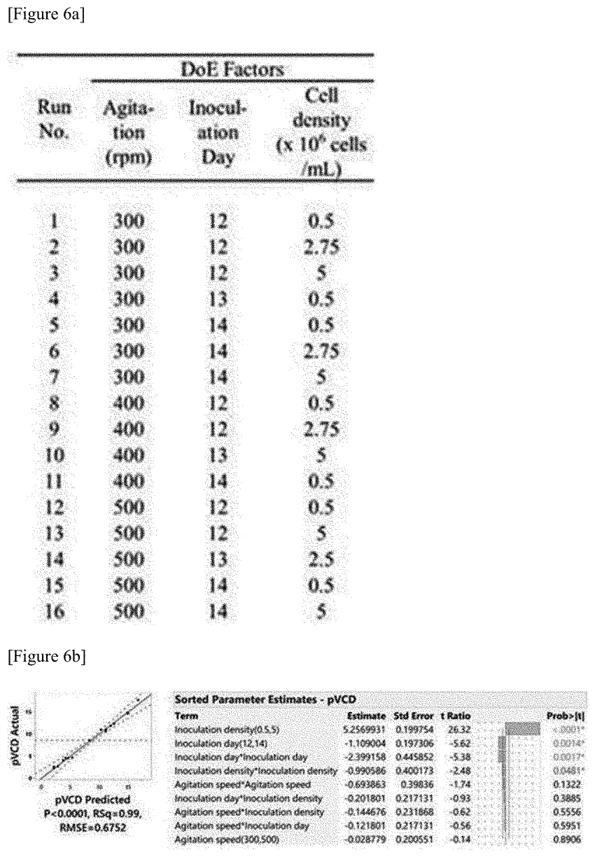

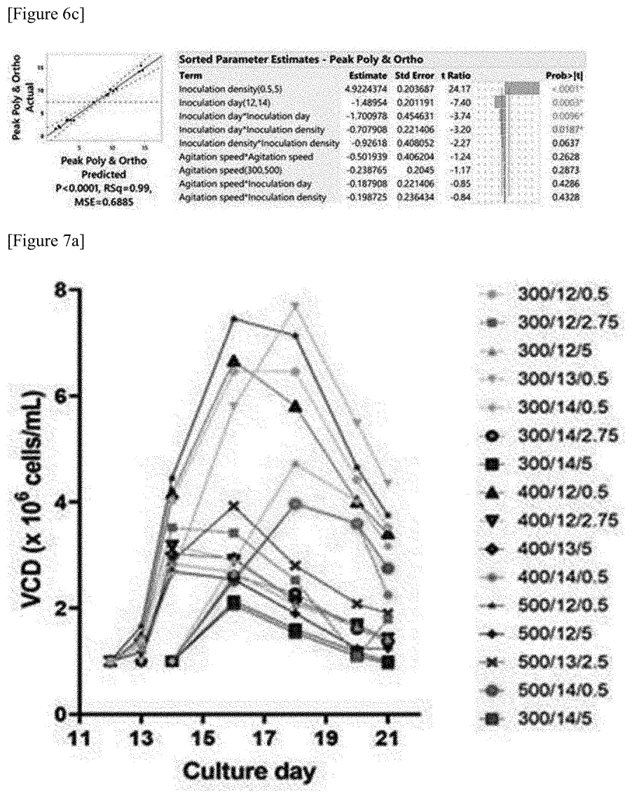

Cell culture process parameters and DoE conditions are shown in FIG. 6A. The graph in FIG. 6B shows the fit and significance analysis of the DoE model for the peak viable cell density (pVCD) during the culture period and the significant values of each parameter, and C shows the fit and significance analysis of each DoE model for the conditions showing the highest cell numbers of polychromatic and orthochromatic erythroblasts and the significant value for each parameter (The blue line is the significance level (95%) and the bar (parameter) above the blue line means a statistically significant factor (p value<0.05).). In the graph in FIG. 6D, the optimal values of the three process factors on the x-axis and the process results on the y-axis are interpreted as the intersection values of the red dotted lines. During the evaluation of the correlation between process results and process factors, when analyzing the time point (culture day) when stationary culture (2D culture) is changed to agitation culture, on day 12.5 of culture after the isolation of hematopoietic stem cells (that is, day 12 or day 13 of culture), the VCD value and the production rate of polychromatic erythroblasts and orthochromatic red blood cells were maximal at a time point before the cell size decreased to 12 μm or less (FIG. 6D). The optimal values for the process factors at the growth phase were the best when the agitation speed was 450 to 500 rpm, the time point of changing from stationary culture (2D culture) to agitation culture was days 12 and 13 of culture, and the inoculation cell concentration was 5×10⁶ cells/ml (FIG. 6D). Based on day 21 of culture (the day of cell harvesting), the model effectiveness of the experimental values of three process factors (agitation speed, inoculation day, and inoculation density) and the process results (viable cell density (VCD), cell viability and cell diameter) was validated, and these correlations were evaluated to derive the optimal value of the process (FIG. 6D).

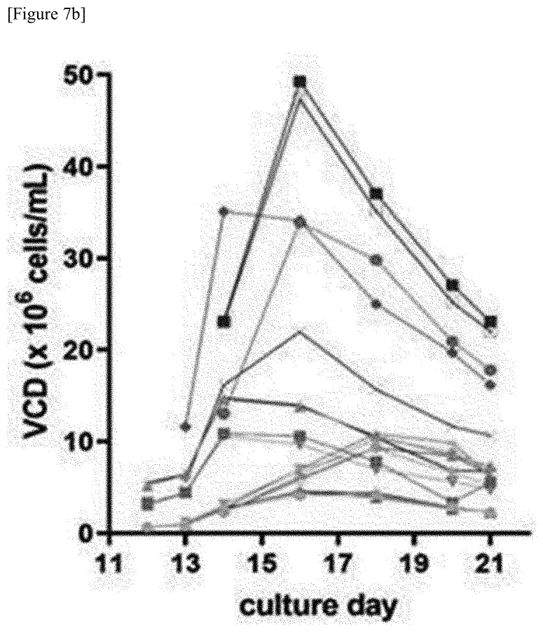

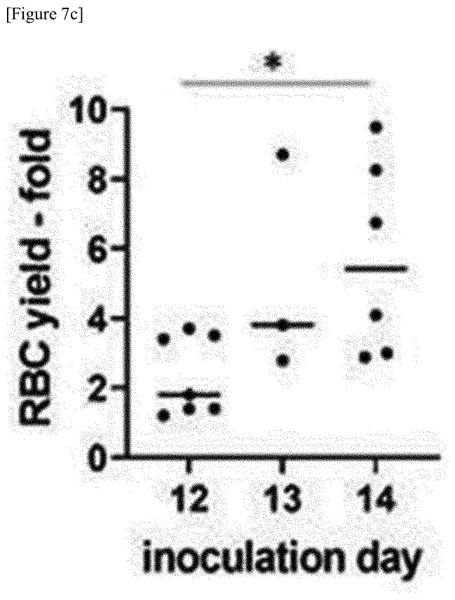

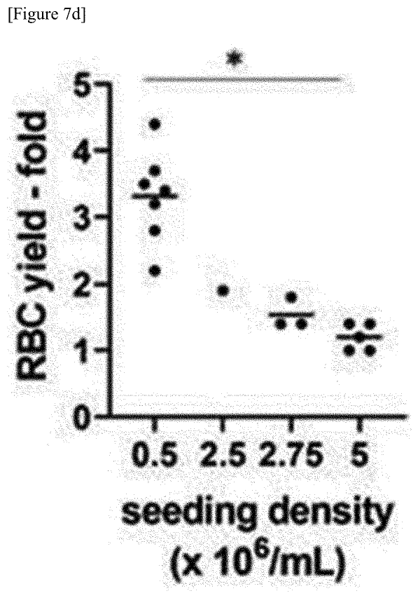

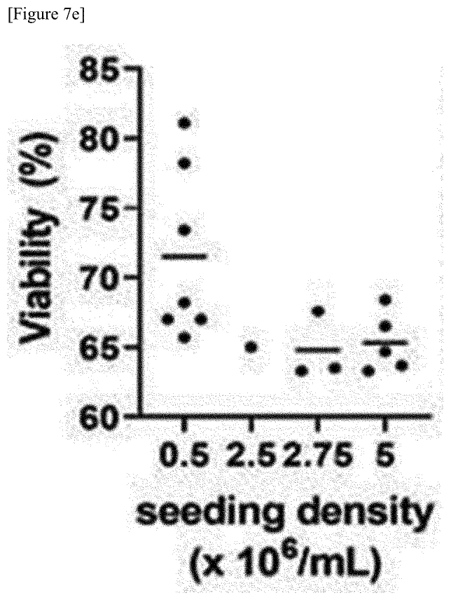

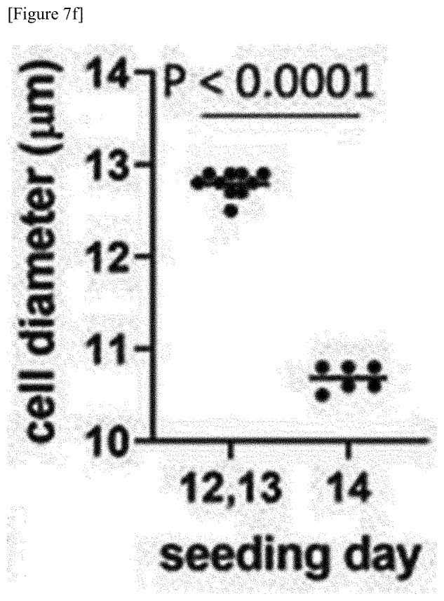

However, this optimal value is a value compared regardless of the culture period in the bioreactor after inoculation (FIG. 7A), and in actual blood production, the amount of actually obtained red blood cells needs to be evaluated in consideration of the actual cell proliferation rate up to the time of inoculation. Accordingly, when the amount of red blood cells obtained is calculated again in consideration of the degree of proliferation of erythroid cells before inoculation into the bioreactor (agitation culture) in the stationary culture (2D culture) (2-fold on days 12 to 13 and 2-fold on days 13 to 14), as a result, it could be seen that significantly more red blood cells were obtained on day 14, which is late inoculation, compared to day 12, because immature cells grew better in stationary culture before inoculation (FIGS. 7B and 7C; ANOVA analysis, Kruskal-Wallis multiple comparison test, *P value<0.05). Further, the group with a higher cell inoculation concentration inevitably has a large amount of final red blood cells. Accordingly, in order to compare the relative red blood cell yield for each culture environment, the final amount of obtained red blood cells was divided by the seeding cell number. As a result, considering that the red blood cell yield is significantly higher at a cell density of 0.5×10⁶ cells/mL than at 5×10⁶ cells/mL, it could be seen that a low-concentration culture environment is relatively more advantageous in terms of yield than a high-concentration culture environment even though the culture conditions such as pH, dissolved oxygen, and CO₂ concentration in the bioreactor are maintained (FIG. 7D; ANOVA analysis, Kruskal-Wallis multiple comparison test, *P value<0.05). Furthermore, cell viability was also shown to be high at low concentrations (FIG. 7E). However, since the difference was not significant compared to high concentrations, it was shown that cells can be efficiently produced even at high concentrations by adjusting other parameters. In addition, there was no change in cell viability according to rpm.

Meanwhile, the cell size on inoculation days 12 and 13 of inoculation into the bioreactor was 12.79±0.13 μm, and the cell size on inoculation day 14 was 10.68±0.13 μm, showing a significant difference (FIG. 7F, mean±standard deviation; unpaired t-test). When combined with the previous results, it could be seen that inoculating the cells into the bioreactor and initiating agitation culture when the cell size was 12 to 13 μm was advantageous in terms of the yield of red blood cells.

### Example 3 Functional Evaluation of Red Blood Cells Produced by Present Invention

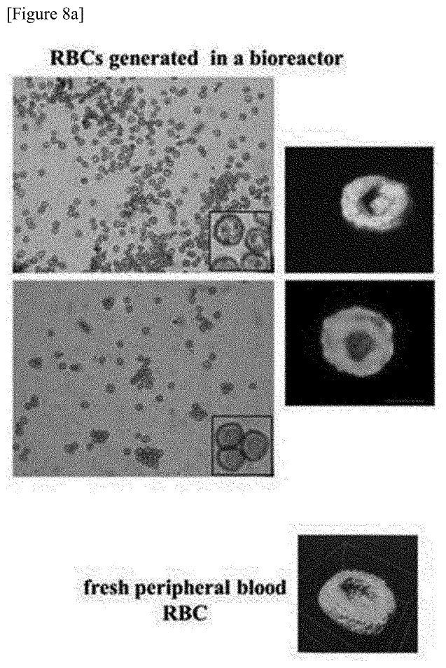

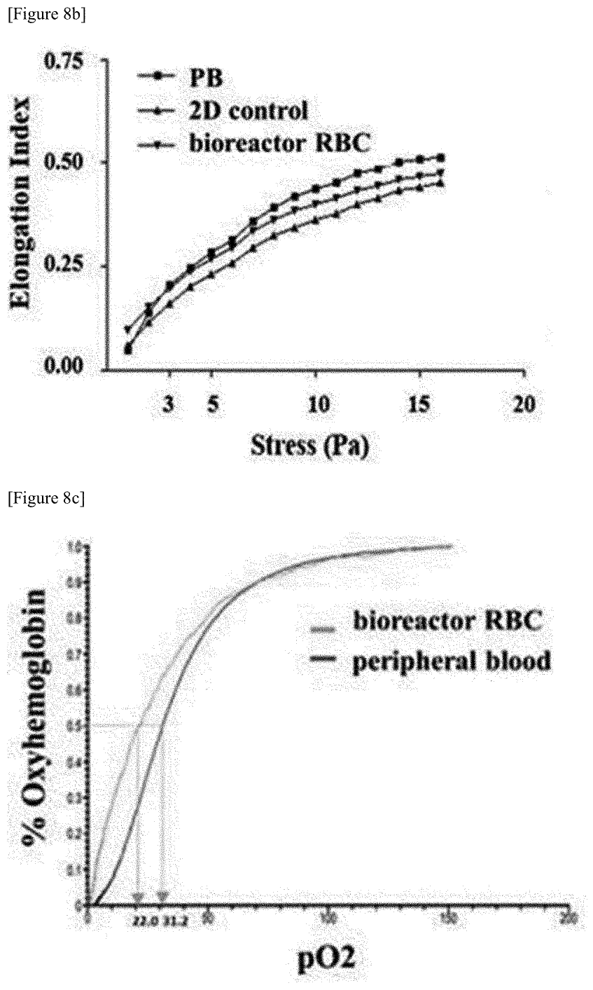

The functional evaluation of red blood cells produced according to the present invention was performed after harvesting cells cultured in a continuously stirred-tank bioreactor (Ambr) on day 21. After only pure red blood cells were filtered through a filter (FIG. 8A; n=2), red blood cell deformability was measured and compared by applying pressure to red blood cells in order to verify whether the red blood cells can withstand the blood flow well without being broken in the same manner as in the peripheral blood of a healthy person (FIG. 8B). The deformability of red blood cells according to pressure was analyzed by passing a laser through them and capturing images of the flowing red blood cells with a camera to analyze the shape of the red blood cells. As the pressure decreased over time, the shape of red blood cells changed from elliptical to spherical, and in this case, the red blood cell elongation index (EI) was calculated based on the pressure of 3 pascals (Pa) (RheoScan-D200, SEWON Meditech, KR).

As a result, the EI values of peripheral blood of a healthy person and produced red blood cells were 0.31% and 0.29%, respectively, based on the pressure of 3 Pa, showing no difference in deformability, and exhibited better values than red blood cells produced by 2D culture (FIG. 8B).

Furthermore, in order to verify the oxygen-carrying capacity, p50 values were measured and compared with those of the peripheral blood of a healthy person as a control from oxygen equilibrium curves using a Hemox analyzer (TCS Scientific) (FIG. 8C).

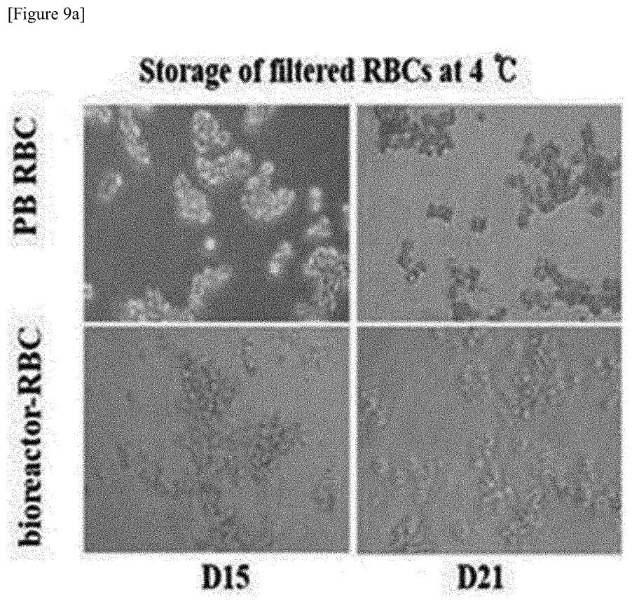

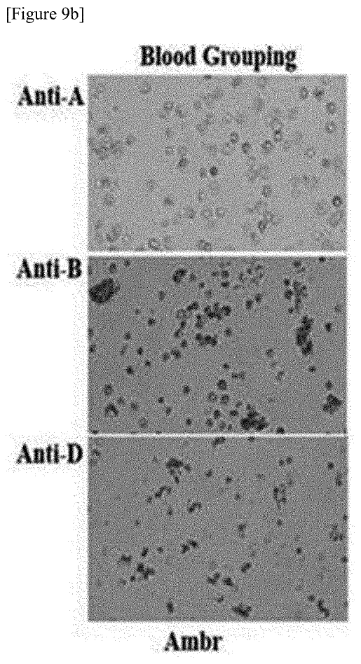

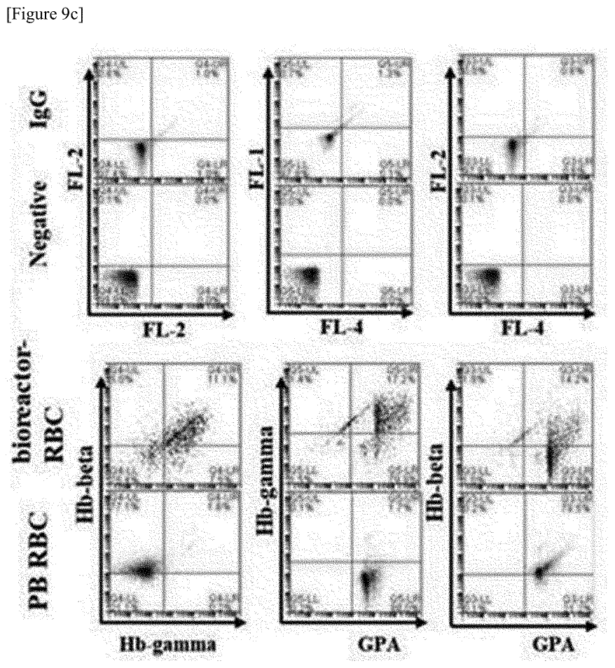

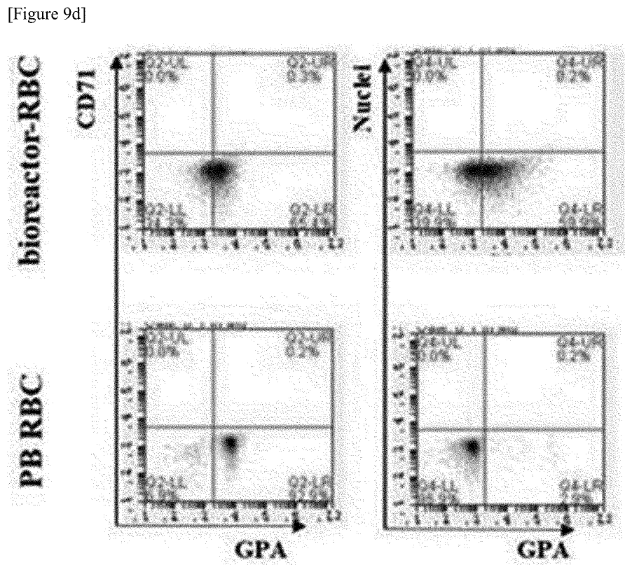

As a result, it was confirmed that the oxygen-carrying capacity of the red blood cells produced according to the present invention appeared to be similar to that of the peripheral blood control of a healthy person (red blood cells produced according to the present invention p50=24.9; control p50=30.9). The red blood cells produced according to the present invention were successfully stored in a refrigerator for 21 days and most of the red blood cells maintained their biconcave red blood cell shape well (FIG. 9A). Further, it was confirmed that a blood type test can be performed (FIG. 9B). Additionally, red blood cells produced according to the present invention expressed hemoglobin (Hb) equivalent to red blood cells produced by 2D culture (FIG. 9C; glycophorin A (GPA) is a marker specific for erythroid cells; Hb-gamma is fetal hemoglobin; and Hb-beta is adult hemoglobin). Furthermore, fluorescent antibodies against glycophorin A (GPA), CD71 and nuclei were attached to red blood cells (bioreactor-RBC) produced in the bioreactor to perform flow cytometry (FIG. 9D). As a result, it was found that the red blood cells had a GPA+/CD71−/nuclei-type similar to that of fresh peripheral blood red blood cells (PB RBC). Through this, it could be confirmed that the red blood cells according to the present invention correspond to mature red blood cells.

1. A method for in vitro production of red blood cells, comprising the step of, during culture of erythrocyte progenitor cells, switching the cultured cells to agitation culture when the diameter of the erythrocyte progenitor cells reaches 10 to 15 μm.

2. The method of claim 1, wherein stationary culture is performed until the diameter of erythrocyte progenitor cells reaches 10 to 15 μm.

3. The method of claim 2, wherein during stationary culture in a bioreactor or an agitator, the stationary culture is performed under the conditions of a hydrodynamic shear stress of substantially less than 30 rpm or a tip speed of less than 0.018 m/s at a temperature of 20 to 38° C.

4. The method of claim 1, wherein the agitation culture is performed under the conditions of a hydrodynamic shear stress of 200 rpm to 800 rpm due to the flow of the medium or a tip speed of 0.15 m/s to 0.48 m/s.

5. The method of claim 1, wherein the agitation culture increases the cell density of erythroid cells by smoothly supplying oxygen and nutrients through agitation of the medium.

6. The method of claim 1, wherein the agitation culture is for obtaining fully mature red blood cells.

7. The method of claim 1, wherein the erythrocyte progenitor cells are cultured in a stroma-free medium.

8. The method of claim 1, wherein the erythrocyte progenitor cells are erythroid cells before enucleation.

9. The method of claim 1, wherein the erythrocyte progenitor cells are proerythroblasts, basophilic erythroblasts, polychromatic erythroblasts, orthochromatic erythrocytes, or mixtures thereof.

10. The method of claim 1, wherein the stationary culture or agitation culture is performed in a bioreactor or an agitator.

11. A blood preparation comprising red blood cells produced by the method of claim 1.
